(19) United States
(12) Patent Application Publication
(10) Pub. No.: US 2021/0217485 A1
(43) Pub. Date: Jul. 15, 2021

(54) METHOD OF ESTABLISHING A CORONARY ARTERY DISEASE PREDICTION MODEL FOR SCREENING CORONARY ARTERY DISEASE

(71) Applicants: Jang-Jih Lu, Taipei City (TW); Chun-Hsien Chen, Taoyuan City (TW); Hsin-Yao Wang, Chiayi City (TW); Yi-Hsin Chan, Taipei City (TW); Wei-Shang Shih, Taipei City (TW)

(72) Inventors: Jang-Jih Lu, Taipei City (TW); Chun-Hsien Chen, Taoyuan City (TW); Hsin-Yao Wang, Chiayi City (TW); Yi-Hsin Chan, Taipei City (TW); Wei-Shang Shih, Taipei City (TW)

(73) Assignees: CHANG GUNG MEMORIAL HOSPITAL, LINKOU, Taoyuan City (TW); Chang Gung University, Taoyuan City (TW); CATHAY GENERAL HOSPITAL, Taipei City (TW)

(21) Appl. No.: 17/220,105

(22) Filed: Apr. 1, 2021

Related U.S. Application Data
(63) Continuation-in-part of application No. 15/871,159, filed on Jan. 15, 2018.

(51) Int. Cl. G16B 5/20; G16B 40/00; G16C 20/30; G16C 20/70; G16H 50/50; G16H 50/20

(52) U.S. Cl. CPC G16B 5/20; G16B 40/00; G16H 50/20; G16C 20/70; G16H 50/50; G16C 20/30

(57) ABSTRACT

A method of establishing a coronary artery disease (CAD) prediction model for CAD screening includes establishing a data set in a computer equipment; entering the data set and corresponding future CAD condition of asymptomatic individuals into a machine learning component; selecting a plurality of robust variables from the clinical data and the cardiovascular markers of the cardiovascular markers panel by using feature selection methods; establishing the CAD prediction model by using machine learning methods; uploading new clinical data and new results of the cardiovascular markers to the cloud-based platform when any asymptomatic individuals undergo the health examination, and performing calculation and analysis by the CAD prediction model; and notifying the asymptomatic individuals of having a high risk of encountering a CAD event or not in a certain period of follow-up time.

### CROSS-REFERENCE TO RELATED APPLICATION

The present application is a continuation in part of U.S. patent application Ser. No. 15/871,159, filed on Jan. 15, 2018, titled Coronary Artery Disease Screening Method by Using Cardiovascular Markers and Machine Learning Algorithms, listing Jang-Jih Lu, Chun-Hsien Chen, Hsin-Yao Wang, Yi-Hsin Chan, and Wei-Shang Shih as inventors.

### BACKGROUND OF THE INVENTION

### 1. Field of the Invention

The invention relates to coronary artery disease (CAD) screening methods and more particularly to a method of establishing a coronary artery disease prediction model for screening coronary artery disease.

### 2. Description of Related Art

Deaths related to cardiovascular diseases are very high in many developing and developed countries. In particular, CAD may cause sudden cardiac death owing to acute coronary syndrome. Healing and caring for CAD patients can cause a great financial burden on the society. An early diagnosis of CAD can decrease the possibility of acute coronary syndrome, heart failure and other complications. However, simple CAD screening methods for asymptomatic people are not disclosed in the art. To the contrary, the conventional CAD screening technologies are disadvantageous owing to the factors of time consuming, high cost, radiation exposure, danger and manual determination.

For example, common CAD screening methods for asymptomatic people at risk of CAD include: cardiac nuclear medicine examination, cardiac catheterization and computed tomography coronary angiography. These methods aim to screen out CAD from people having no significant symptom. While these methods are effective, they have limitations. High radiation risk exists in cardiac nuclear medicine examination, cardiac catheterization and computed tomography coronary angiography. Cardiac catheterization has the highest accuracy but it has the risk of penetrating coronary arteries in operation. Computed tomography coronary angiography is a CAD screening method having a low invasiveness and a high accuracy. But it relies on computed tomography coronary angiography. Further, it has the problems of radiation exposure, high cost of equipment for computed tomography coronary angiography, high diagnosis cost and inappropriateness for large scale screening.

Another conventional CAD screening method involves a cardiovascular markers panel including many test values of the cardiovascular markers. Thus, a manual reading of the test values by a medical employee is required. The reading and interpretation of the test values are based on the threshold values of the cardiovascular markers. That is, a person being diagnosed may have a high risk of having CAD if the test value of any cardiovascular marker is greater than its corresponding threshold value. However, such method does not consider the comprehensive data distribution pattern of the cardiovascular markers as a whole. And in turn, it is not accurate and has a low performance in clinical use.

It is concluded that these conventional CAD screening methods are disadvantageous due to the drawbacks of inconvenience, high cost, and exposure to medical related damage and radiation.

Thus, the need for a practical, convenient and safe method for screening CAD of ordinary people having no CAD symptom still exists.

### SUMMARY OF THE INVENTION

Therefore one object of the invention is to provide a method of establishing a CAD prediction model to screen CAD for asymptomatic individuals. The method comprises the following steps: a). establishing a data set in a computer equipment, wherein the data set is clinical data obtained from a plurality of asymptomatic individuals undergoing health examination, and test results of a plurality of samples from the asymptomatic individuals by using a cardiovascular markers panel including a plurality of cardiovascular markers; b). entering the data set and corresponding future CAD conditions of the asymptomatic individuals into a machine learning component wherein the machine learning component is established in a cloud-based platform provided for data upload and download, thereby new data set is continuously entered into the machine learning component to enhance learning c). selecting a plurality of robust variables from the clinical data and the cardiovascular markers of the cardiovascular markers panel by using feature selection methods; d). establishing the CAD prediction model by using machine learning methods; e). uploading new clinical data and new test results of the cardiovascular markers to the CAD prediction model when any asymptomatic individuals undergo the health examination, and performing calculation and analysis by the CAD prediction model, wherein the CAD prediction model anticipates future CAD risk of the asymptomatic individuals; f). notifying an individual of having a high risk of encountering a CAD event within a certain period of follow-up time by sending messages from the cloud-based platform when the determination of step f is positive, wherein the messages include suggestions on medical interventions, better exercise, diet, and daily routine to lower the risk of encountering the CAD event within the certain period of follow-up time.

The above and other objects, features and advantages of the invention will become apparent from the following detailed description taken with the accompanying drawings.

### BRIEF DESCRIPTION OF THE DRAWINGS

FIG. 1 is a flowchart of the CAD screening method according to the invention; and

FIG. 2 is a chart showing CAD prediction performance by using single cardiovascular marker or a cardiovascular markers panel combined with machine learning methods in terms of the area under the receiver operating characteristic (ROC) curve.

### DETAILED DESCRIPTION OF THE INVENTION

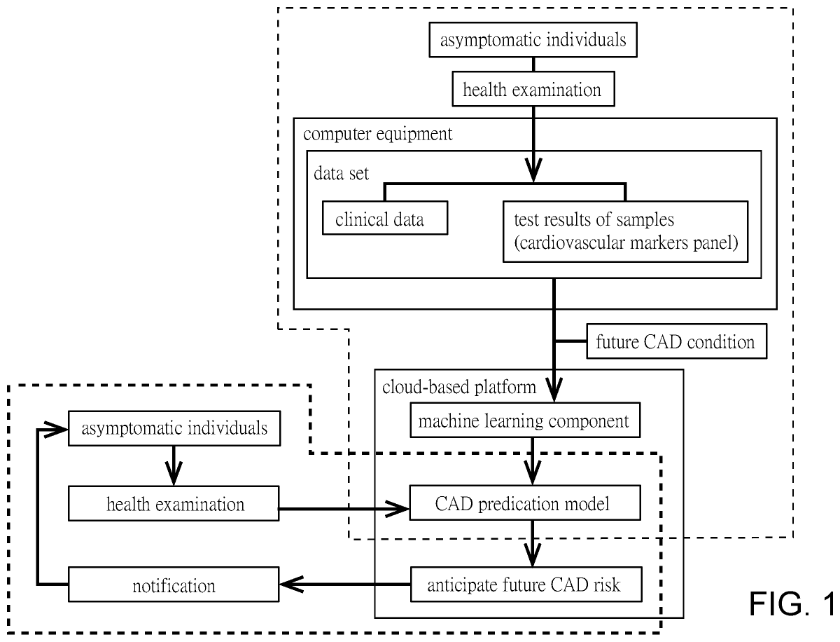

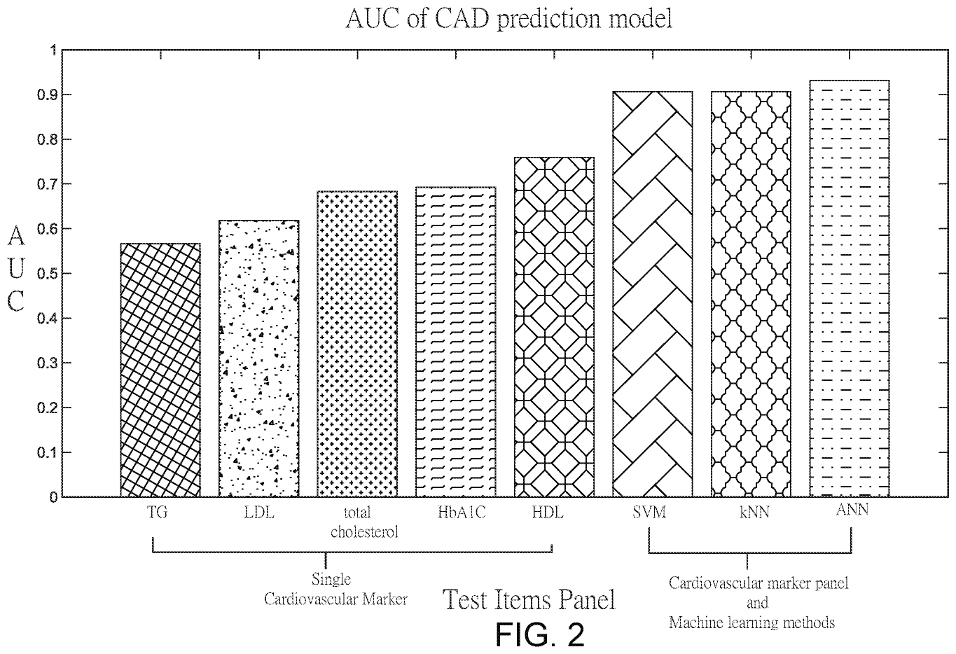

Referring to FIGS. 1 and 2, a CAD prediction model established in accordance with the invention comprises the following steps as described in detail below.

First, establish a data set in a computer equipment, wherein the data set is clinical data obtained from a plurality of asymptomatic individuals undergoing health examination, and test results of a plurality of samples from the asymptomatic individuals by using a cardiovascular markers panel including a plurality of cardiovascular markers. Clinical data of the asymptomatic individuals including sex, age, Body Mass Index (BMI), hypertension status, as well as diabetes mellitus status are collected, and samples such as blood, urine, saliva, sweat, feces, pleural fluid, and ascites fluid or cerebrospinal fluid of the individuals are tested by using a cardiovascular markers panel. Next, enter the data set and corresponding future CAD condition of the asymptomatic individuals into a machine learning component. The machine learning component is established in a cloud-based platform provided for data upload and download, so new data set is continuously entered into the machine learning component to enhance learning. Select a plurality of robust variables from the clinical data and the cardiovascular markers of the cardiovascular markers panel by using feature selection methods. Establish the CAD prediction model by using machine learning methods. Next, upload new clinical data and new test results of the cardiovascular markers to the CAD prediction model when any asymptomatic individuals undergo the health examination, and perform calculation and analysis by the CAD prediction model. As a result, the CAD prediction model anticipates future CAD risk of the asymptomatic individuals. Last, notify an individual of having a high risk of encountering a CAD event within a certain period of follow-up time by sending messages from the cloud-based platform when the determination of the CAD prediction model is positive, wherein the messages include suggestions on medical interventions, better exercise, diet, and daily routine to lower the risk of encountering the CAD event within the certain period of follow-up time.

The individuals may not take the health examination at the same hospital every time. Thus, sometimes the hospital may not have prior data from another hospital and can only compare the results of the individuals with public data. However, according to the invention, the machine learning component established in the cloud-based platform provides different hospitals to upload examination data of all the individuals, and integrating it to form big data. Therefore, the individuals are not limited to taking the health examination at the same hospital every time. Further, by a sharing of big data, medical personnel can enter more data to enhance the learning of the machine learning component. As a result, the CAD prediction model established by the machine learning component becomes more precise on anticipating CAD risk of the asymptomatic individuals.

The corresponding future CAD condition is classified as having CAD or not. When the CAD event occurred to the asymptomatic individual within the certain period of follow-up time after the health examination and the individual was being diagnosed as having CAD by a doctor using gold standard, the corresponding future CAD condition of the asymptomatic individual is classified as having CAD, otherwise classified as not having CAD. The certain period of follow-up time is any length of time ranging from a day to three years. The gold standard mentioned above is the present way of diagnosing CAD, which is cardiac catheterization and angiography of coronary artery with the highest accuracy.

The cardiovascular markers panel includes High Density Lipoprotein (HDL), Low Density Lipoprotein (LDL), Triglycerol (TG), total cholesterol, blood sugar, micro-albumin, glycosylated hemoglobin (HbA1C), High-Sensitivity C-Reactive Protein (hsCRP), Homocysteine, lipoprotein, uric acid, cardiac troponins, creatine kinase (CK), N-terminal Pro Brain Natriuretic Peptide (NT ProBNP), B-type Natraretic Peptide (BNP), N-terminal Pro Brain Natriuretic Peptide (NT ProBNP), procalcitonin (PCT), erythrocyte sedimentation rate (ESR), lactic dehydrogenase (LDH), Na⁺, K⁺, Ca²⁺, Mg²⁺, Fe2+, Fe³+, Urea Nitrogen, Creatinine, Cystatin C, Bilirubin, Ketone and pH.

An embodiment is detailed below.

Conditions (including admission and exclusion) of an individual being screened and the number of samples:

An adult of at least 20-year old is appropriate for taking the test of the cardiovascular markers panel. Medical records of patients are checked to find 543 potential candidates. Thus, there is no need of recruiting candidates.

Design and Method:

Clinical data, test items and measurements include sex, age, Body Mass Index (BMI), Hypertension status, Diabetes mellitus status, High Density Lipoprotein (HDL), Low Density Lipoprotein (LDL), Triglycerol (TG), and glycosylated hemoglobin (HbA1C). There are 543 candidates and blood drawing and cardiac catheterization are conducted on each candidate in order to determine their CAD state.

Feature selection: after preliminary data cleaning, a univariate statistics is conducted in the embodiment. An appropriate univariate statistics (e.g., Chi-square test or t test) is selected based on the characteristic of the variables. As a result, variables including sex, BMI, diabetes mellitus status, hypertension status, TG, low density lipoprotein, total cholesterol, HbA1C and high density lipoprotein are selected as features of subsequent model training.

However, univariate statistics belong to filter methods for variable selection. Wrapper methods, embedded methods, and other filter methods can also be applied to the selection of robust variables from the clinical information and optimum cardiovascular markers of the cardiovascular markers panel.

After the feature selection, a plurality of CAD prediction models are established by machine learning methods in the embodiment, and the machine learning methods include k-nearest neighbors, k Nearest Neighbor (kNN), Support Vector Machines (SVM) and

Artificial Neuron Network (ANN).

Retrospective period of the embodiment: from Sep. 1, 2010 to Mar. 31, 2011.

Result evaluation and statistical method:

In the embodiment, data distributions of the cardiovascular markers are calculated. Further, prediction models are trained based on the selected variables and their values. In the embodiment, 5-fold cross-validation is used to evaluate the prediction performance of each prediction model. Performance of the prediction model is evaluated based on the ROC curve and the area under the curve (AUC) is calculated accordingly.

FIG. 2 is a chart showing the CAD prediction performance of various prediction models in terms of AUC. The AUCs of CAD prediction models established by single cardiovascular markers (namely, TG, low density lipoprotein, total cholesterol, HbA1C or high density lipoprotein) and the AUCs of CAD prediction models established by the cardiovascular markers panel combined with different machine learning methods(namely, SVM, kNN or Artificial Neural Network) are used to evaluate the CAD prediction performance. From the figure, it is shown that the AUC of the prediction model using a single cardiovascular marker is about 0.7 at most. However, for a prediction model using one of the machine learning methods to analyze the cardiovascular markers panel (including a plurality of cardiovascular markers), the CAD prediction AUC can be greatly increased to about 0.9. Thus, using machine learning methods to integrate and learn the data of the cardiovascular markers panel can greatly increase the performance of CAD screening.

It is concluded that the invention has the following characteristics and advantages: The cardiovascular markers panel can obtain test results of a plurality of cardiovascular markers in a single blood test for asymptomatic individuals being screened for CAD. Integrating clinical data and the test data of the cardiovascular markers with machine learning methods allows comprehensive analysis of the distribution difference between CAD and non-CAD cases. The trained CAD prediction model can be easily copied to users' computers for use. Thus, it can be widely used in CAD screening. Therefore, it contributes greatly to the advancement of medical diagnosis. Further, its accuracy, time efficiency, cost effectiveness and repeatability in comparison with the conventional manual reading methods are greatly improved. Further, invasiveness and risk of radiation exposure are greatly decreased compared to the conventional CAD screening methods.

While the invention has been described in terms of preferred embodiments, those skilled in the art will recognize that the invention can be practiced with modifications within the spirit and scope of the appended claims.

What is claimed is:

1. Method of establishing a coronary artery disease (CAD) prediction model for screening CAD comprising the steps of:
(a) establishing a data set in a computer equipment, wherein the data set is clinical data obtained from a plurality of asymptomatic individuals undergoing health examination, and test results of a plurality of samples from the asymptomatic individuals by using a cardiovascular markers panel including a plurality of cardiovascular markers;
(b) entering the data set and corresponding future CAD conditions of the asymptomatic individuals into a machine learning component, wherein the machine learning component is established in a cloud-based platform provided for data upload and download, thereby new data set is continuously entered into the machine learning component to enhance learning;
(c) selecting a plurality of robust variables from the clinical data and the cardiovascular markers of the cardiovascular markers panel by using feature selection methods;
(d) establishing the CAD prediction model by using machine learning methods;
(e) uploading new clinical data and new test results of the cardiovascular markers to the CAD prediction model when any asymptomatic individuals undergo the health examination, and performing calculation and analysis by the CAD prediction model, wherein the CAD prediction model anticipates future CAD risk of the asymptomatic individuals;
(f) notifying an individual of having a high risk of encountering a CAD event within a certain period of follow-up time by sending messages from the cloud-based platform when the determination of step (e) is positive, wherein the messages include suggestions on medical interventions, better exercise, diet, and daily routine to lower the risk of encountering the CAD event within the certain period of follow-up time.

2. The method of claim 1, wherein in step (b) the corresponding future CAD conditions is classified as having CAD or not, when the CAD event occurred to the asymptomatic individual within the certain period of follow-up time after the health examination and the individual was being diagnosed as having CAD by a doctor using gold standard, the corresponding future CAD conditions of the asymptomatic individual is classified as having CAD, otherwise classified as not having CAD;
wherein in step (f) the certain period of follow-up time is any length of time ranging from a day to three years.

3. The method of claim 1, wherein the cardiovascular markers panel includes High Density Lipoprotein (HDL), Low Density Lipoprotein (LDL), Triglycerol (TG), total cholesterol, blood sugar, microalbumin, glycosylated hemoglobin (HbA1C), High-Sensitivity C-Reactive Protein (hsCRP), Homocysteine, lipoprotein, uric acid, cardiac troponins, creatine kinase (CK), N-terminal Pro Brain Natriuretic Peptide (NT ProBNP), B-type Natraretic Peptide (BNP), N-terminal Pro Brain Natriuretic Peptide (NT ProBNP), procalcitonin (PCT), erythrocyte sedimentation rate (ESR), lactic dehydrogenase (LDH), Na+, K+, Ca2+, Cl−, Mg2+, Fe2+, Fe3+, Urea Nitrogen, Creatinine, Cystatin C, Bilirubin, Ketone and pH.

4. The method of claim 1, wherein in step (c) the selection of the robust variables from the clinical data and optimum cardiovascular markers of the cardiovascular markers panel is done by univariate statistics.

5. The method of claim 4, wherein the univariate statistics are Chi-square test and t-test.

6. The method of claim 1, wherein in step (c) the optimum selected cardiovascular marker variables are sex, age, Body Mass Index (BMI), hypertension status, diabetes mellitus status, TG, High Density Lipoprotein (HDL), Low Density Lipoprotein (LDL), total cholesterol, and glycosylated hemoglobin(HbA1C).

7. The method of claim 1, wherein in step (a) the clinical data is including sex, age, Body Mass Index (BMI), hypertension status, and diabetes mellitus status.

8. The method of claim 1, wherein in step (a) the samples are the body fluids includes blood, urine, saliva, sweat, feces, pleural fluid, and ascites fluid or cerebrospinal fluid.

9. The method of claim 1, wherein the machine learning methods are Logistic Regression, k-Nearest Neighbor, Support Vector Machine, Artificial Neural Network, Decision Tree, Random Forest, Bayesian Network, or any combinations thereof.

10. The method of claim 1, wherein in step (c) the selection of the robust variables from the clinical data and optimum cardiovascular markers of the cardiovascular markers panel is done by filter methods, wrapper methods or embedded methods.
